(19) United States
(12) Patent Application Publication
(10) Pub. No.: US 2015/0164858 A1
(43) Pub. Date: Jun. 18, 2015

(54) COMBINATION COMPRISING PARTHENOLIDE FOR USE IN THE TREATMENT OF ALZHEIMER'S DISEASE AND OTHER NEURODEGENERATIVE DISORDERS

(71) Applicant: King Abdullah University of Science and Technology, Thuwal (SA)

(72) Inventors: Vladimir Bajic, Thuwal (SA); Magbubah Essack, Thuwal (SA)

(21) Appl. No.: 14/402,385

(22) PCT Filed: May 22, 2013
(86) PCT No.: PCT/IB2013/001806

§ 371 Date: Nov. 20, 2014

Related U.S. Application Data
(60) Provisional application No. 61/649,964, filed on May 22, 2012.

(51) Int. Cl. A61K 31/365; A61K 31/235; A61K 31/138; A61K 31/12

(52) U.S. Cl. CPC A61K 31/365; A61K 31/12; A61K 31/235; A61K 31/138

(57) ABSTRACT

The present invention generally concerns particular methods and compositions for treatment of a neurodegenerative disease, such as Alzheimer's Disease. In particular embodiments, there is a composition comprising Parthenolide and a second agent, including an inhibitor of TLR4/MD-2/CD14, nAChR agonist, Resatorvid, Curcumin, Tilorone or a Tilorone analog, or a combination thereof.

This application claims priority to U.S. Provisional Patent Application Ser. No. 61/649,964, filed May 22, 2012, which is incorporated by reference herein in its entirety.

### TECHNICAL FIELD

The present invention generally concerns at least the fields of cell biology, molecular biology, and medicine. In particular aspects, the field of the present invention include treatment and/or prevention of neurodegenerative disorders (NDDs) in a mammal.

### BACKGROUND

NDDs are hereditary and sporadic conditions which are characterized by progressive nervous system dysfunction (Bredesen, et al 2006). NDDs include diseases such as Alzheimer's disease (AD), Parkinson's disease (PD), Multiple Sclerosis (MS), Amyotrophic Lateral Sclerosis (ALS or Lou Gehrig's disease), Huntington's disease, Prion diseases, and others (Ekshyyan and Aw 2004). For example, Alzheimer's disease (AD) is the most common type of dementia and is associated with progressive loss of mental activities and memory (Salawu, et al 2011). The nature of the neurodegeneration in AD suggests an age-dependent process that ultimately leads to dendritic and neuronal damage throughout the brain (Isacson, et al 2002). This fact is highlighted in the 2011 Alzheimer's Disease Facts and Figures in the USA, as an estimated 5.2 million people aged 65 and older have AD and 200,000 individuals under age 65 who have early-onset AD, with an associated healthcare cost in excess of US$183 billion annually (http://www.alz.org/downloads/Facts_Figures_{—}2011.pdf).

AD is the most common type of dementia and is associated with progressive loss of mental processes and memory (Salawu, et al 2011). Mutation of the Apolipoprotein E (ApoE) gene appears to be a major genetic susceptibility risk factor for the development of typical late-onset AD (Liu, et al 2007). In contrast to late onset AD, early onset AD or familial AD is rare and is inherited due to genetic mutations of the presenilin 1 (PS-1) and presenilin 2 (PS-2) genes accompanied by mutations of the amyloid precursor protein (APP) gene (Scheuner, et al 1996). The APP gene codes for the amyloid β (Aβ) peptides that are the primary component of senile plaques (Goedert and Spillantini 2006). AD arises from the Aβ peptides triggering neuronal cell death. However, the development and progression of AD is not only influenced by the gene's effect on amyloid plaque and intracellular tangle formation but also by environmental factors, such as cytokines and neurotoxins.

AD is characterized by neuronal loss of the superficial cortex and synaptic alterations, such as reduction of pre-synaptic terminal density (Cummings, et al 1998). Microscopically, the two identifying cardinal features of AD are amyloid plaques and neurofibrillary tangles. The prevailing model for AD causation is the so-called “amyloid hypothesis” that ascribes a causative role in AD to abnormal amyloid processing and deposits (Hardy and Selkoe 2002). It has been demonstrated that a decrease in the neurotransmitter acetylcholine (Ach) has a direct impact on memory loss, and thus the loss of cholinergic neurons may underlie memory loss in AD (Babic 1999). It has also been demonstrated that the progression of AD is further complemented with glutaminergic, noradrenergic, and serotonergic system deficiencies that deteriorates cognitive and memory loss. Modest success in improving AD symptoms has been achieved with therapeutics (such as memantine, galantamine, rivastigmine and donepezil) that focuses on correcting neurotransmitter deficits.

It has also been demonstrated that levels of cholinergic activity and neuronal nicotinic acetylcholine receptor (nAChR) activity decreases with disease progression of AD. The AChR subtypes implicated in the progression of AD include the α4 and α7 containing nAChR subtypes (Liu, et al 2009, Mousavi, et al 2003). This finding has been supported by recent studies that demonstrate that Aβ peptides can directly and indirectly affect nAChR-mediated synaptic transmission (Srivareerat, et at 2011) and that nAChR agonists increase sAPPα secretion whilst decreasing levels of Aβ peptides (Mousavi and Hellstrom-Lindahl 2009).

The mechanism by which Aβ peptides induce the neuronal cell death is not clear. However, numerous mechanisms such as intracellular calcium accumulation, reactive oxygen species (ROS) and nitric oxide (NO) productions, alteration of the cytoskeleton and nucleus and inflammatory processes that converge to the ubiquitous pathways of necrosis or apoptosis have been proposed. Since the AD brain is characterized by an ongoing chronic inflammatory process, research is directed at finding the root of this inflammatory response. It has been demonstrated that specialized cells in the brain such as astrocytes and microglia are increased in the brain of AD patients (Itagaki, et al 1989). Reactive astrocytes showed increased levels of phospholipase A2 that induces increased activity in the arachidonic acid/prostaglandin inflammatory pathway (Moses, et al 2006). However, recent findings suggest that Aβ is indirectly neurotoxic by activating microglia to produce ROS (Parihar and Hemnani 2004). Moreover, it has also been demonstrated that activated microglial cells express neurotoxic compounds (including superoxides, glutamate and NO) (Brown and Bal-Price 2003, Marzolo, et al 1999) and secrete interleukin-1 (IL-1), natural killer and antigen presenting cells (Giulian 1987). It has additionally been shown that cytotoxic and helper T lymphocyte infiltration enhance the levels of major histocompatibility complex (MHC) glycoproteins on activated microglia (Rogers, et al 1992) that is associated with compact senile plaques.

Cytokines that control the recruitment of lymphocytes to the sites of inflammation, has also been reported in senile plaques (Griffin, et al 1989). Moreover, it has been demonstrated that levels of IL-1 are increased in microglia around diffuse amyloid plaques (Rogers, et al 1999) and that IL-1 increases the translation of the mRNA encoded by APP gene (Colton, et al 1994) leading to early onset of AD.

A recent study by Walter et al. demonstrated that supernatant of amyloid peptide-stimulates microglia and that TLR4 contributes to amyloid peptide-induced microglial neurotoxicity. In addition, stimulation experiments allowed for the identification of a tri-molecular receptor complex consisting of TLR4, MD-2 and CD14 necessary for full cellular activation by aggregated amyloid peptide (Walter, et al 2007). Additionally, Burguillos et al. (2011) demonstrated that pro-inflammatory stimuli induced activation of caspase-8, -3 and -7 in microglia without triggering cell death. The activation of these caspases was further shown to be dependent on TLR4 but independent of MyD88 (Burguillos, et al 2011).

Interestingly, apoptosis after TLR2 activation has been demonstrated to be associated with the formation of a myeloid differentiation factor 88 (MyD88)/Fas-associated death domain protein (FADD)/Caspase-8 complex (Aliprantis, et al 2000). Moreover, Burguillos et al. demonstrated that inhibition of the proinflammatory stimuli induced cascade in microglia presents neurodegeneration in individuals with AD and PD (Burguillos, et al 2011). Thus non-steroidal anti-inflammatory drugs are useful in patients having risk for AD, in at least certain aspects.

The present invention provides a long-felt need in the art to improve neurodegeneration in an individual afflicted thereby.

### BRIEF SUMMARY OF THE INVENTION

In embodiments of the invention, there are methods and compositions related to treatment and/or prevention of neurodegenerative diseases (NDD), including AD, PD, MS, ALS, Huntington's disease, or Prion diseases, for example, and others. In specific aspects of the invention, there are methods and compositions that comprise combinatorial strategies for the treatment and/or prevention of AD. In some embodiments, the individual being treated is known to have a NDD or is at risk for developing a NDD, including known to have AD or is at risk for developing AD, as an example. Individuals at risk for AD include those that are 65 years or older, that have defects in the ApoE gene, have defects in presenilin 1 or presenilin 2, and/or have defects in the APP gene, and so forth.

In certain embodiments of the invention, there are combination therapies and/or combination preventative measures that include Parthenolide (1aR,7aS,10aS,10bS)-1a,5-dimethyl-8-methylene-2,3,6,7,7a,8,10a,10b-octahydrooxireno[9,10]cyclodeca[1,2-b]furan-9(1aH)-one) and one or more other compounds. In specific embodiments, there are the following exemplary combinations: 1) Parthenolide and one or more inhibitors of the tri-molecular receptor complex (TLR4/MD-2/CD14); 2) Parthenolide and Resatorvid; 3) Parthenolide, Resatorvid and Curcumin; 4) Parthenolide, an inhibitor of or inhibitors of the tri-molecular receptor complex (TLR4/MD-2/CD14) and one or more nACHR agonist(s); 5) Parthenolide, Resatorvid and Tilorone (trade name Amixin IC; 2,7-Bis(2-diethylaminoethoxy)fluorine-9-one) (or Tilorone analog(s), such as Tilorone analog R11-567DA (2,8-Bis(dimethylaminoacetyl)dibenzofuran hydrate dihydrochloride), Tilorone analog R11-877DA (2,8-diylbis((N-carbonylmethylene)dimethylamine)dibenzothiophene), and/or Tilorone analog R10,874DA (3,6-Bis[2-(Dimethylamino)ethoxy]-9H-xanthen-9-one dihydrochloride 3,6-Bis[2-(dimethylamino)ethoxy]-9H-xanthen-9-one dihydrochloride)); 6) Parthenolide, Resatorvid, Curcumin and Tilorone (or Tilorone analog(s)); 7) Parthenolide and nACHR agonist(s); 8) Parthenolide and Tilorone (or Tilorone analog(s)); 9) any of the above-listed combinations in combination with one or more other NDDs treatments, including any AD treatment (including for treating one or more symptoms of the NDD); and/or 10) Parthenolide and any known NDD treatment, including any AD treatment (including for treating one or more symptoms of the neurodegenerative disease).

In particular embodiments of the invention, Parthenolide and one or more other compounds act synergistically in the treatment and/or prevention of one or more NDD in an individual, whereas in some embodiments Parthenolide and one or more other compounds act additively in the treatment and/or prevention of one or more NDD in an individual.

In certain embodiments of the invention, the methods and compositions reduce the progression of AD, and in some embodiments the methods and compositions of the invention are effective to treat a larger spectrum of AD patients than is possible now with the symptomatic therapies. In particular embodiments the invention facilitates treatment of the progression of a NDD, and in certain embodiments facilitates treatment of at least one symptom of a NDD. In certain cases the invention is effective for individuals having early onset or familial AD.

In exemplary embodiments, one or more of the following TLR-4/MD-2/CD14 inhibitors are employed: Atorvastatin (Ajamieh, et al 2012), Betulinic acid (Wan, et al 2012), Follistatin-related protein (FRP) (Murakami, et al 2012), Hyaluronic acid (Riehl, et al 2012), Inulin (Nagahara, et al 2011), Dienogest (Mita, et al 2011), Lactoferrin (LF) (Puddu, et al 2011), Fumigaclavine C (FC) (Du, et al 2011), Vasoactive intestinal peptide (VIP) (21693218), MD2-I (Liu, et al 2011), Long chain n-3 PUFA (EPA and DHA) (Ibrahim, et al 2011), Helenalin (Zhao, et al 2011), Cinnamaldehyde (Zhao, et al 2011), Sulforaphane (Zhao, et al 2011), Eritoran tetrasodium (Tidswell and LaRosa 2011), Kaempferol (Park, et al 2011), Rosiglitazone (Wu, et al 2011), Hydroxyethyl starch (HES) (Tian, et al 2011), Triptolide (Yu, et al 2011), Ferulic acid (FA) (Kim, et al 2011), Baicalin (Tu, et al 2011), Zileuton (Tu, et al 2010), Isoliquiritigenin (Park and Youn 2010), 6-shogaol (Ahn, et al 2009), Acrolein (Lee, et al 2008), E5564 (Kitazawa, et al 2010), Chalcone (Roh, et al 2010), Chalcone derivative: 2′,4-dihydroxy-6′-isopentyloxychalcone (JSH) (Roh, et al 2010), MDMP (Duan, et al 2010), and/or 9,10-Dihydro-2,5-dimethoxyphenanthrene-1,7-diol (RSCL-0520) (Datla, et al 2010).

In some embodiments, one or more of the following nACHR agonists are employed: 5-(4-acetyl[1,4]diazepan-1-yl)pentanoic acid [5-(4-methoxyphenyl)-1H-pyrazol-3-yl]amide (25, SEN15924, WAY-361789) (Zanaletti, et al 2012), SEN12333 (Roncarati, et al 2009), SEN12333/WAY-317538 (Haydar, et al 2009), 4-(5-methyloxazolo[4,5-b]pyridin-2-yl)-1,4-diazabicyclo[3.2.2]nonane (24, CP-810,123) (O'Donnell, et al 2010), N-[(3R,5R)-1-azabicyclo[3.2.1]oct-3-yl]furo[2,3-c]pyridine-5-carboxamide (3a, PHA-709829) (Acker, et al 2008), N-[(3R)-1-azabicyclo[2.2.2]oct-3-yl]-7-[2-(methoxy)phenyl]-1-benzofuran-2-carboxamide (ABBF) (Boess, et al 2007), 5-(6-[(3R)-1-azabicyclo[2,2,2]oct-3-yloxy]pyridazin-3-yl)-1H-indole (ABT-107) (Malysz, et al 2010), 4-(5-(4-chlorophenyl)-2-methyl-3-propionyl-1H-pyrrol-1-yl)benzenesulfonamide (A-867744) (Malysz et al 2009), N-[(3R)-1-Azabicyclo[2.2.2]oct-3-yl]furo[2,3-c]pyridine-5-carboxamide (14, PHA-543,613) (Wishka et al 2006), (R)-3′-(3-methylbenzo[b]thiophen-5-yl)spiro[1-azabicyclo[2,2,2]octane-3,5′-oxazolidin]-2′-one (Tatsumi, et al 2006), (R)-3′-(5-Chlorothiophen-2-yl)spiro-1-azabicyclo[2.2.2]octane-3,5′-[1′,3′]oxazolidin-2′-one (Tatsumi, et al 2005), N-[(3R)-1-azabicyclo[2.2.2]oct-3-yl]-4-chlorobenzamide hydrochloride (PNU-282987) (Hajos, et al 2005), WYE-103914/SEN34625 (Marquis, et al 2011) and/or 1-[6-(4-fluorophenyl)pyridin-3-yl]-3-(4-piperidin-1-ylbutyl)urea (SEN34625/WYE-103914) (Ghiron, et al 2010).

In some embodiments, there is a composition comprising Parthenolide and at least a second agent, wherein said second agent is selected from the group consisting of: a) one or more inhibitors of the tri-molecular receptor complex (TLR4/MD-2/CD14); b) one or more nicotinic acetylcholine receptors (nACHR) agonists; c) a neurodegenerative disease treatment; and d) a combination thereof. In specific embodiments, an inhibitor of TLR4/MD-2/CD14 is selected from the group consisting of Curcumin, Resatorvid, and a combination thereof. In some embodiments, a nACHR agonist is selected from the group consisting of Tilorone, Tilorone analog R11-567DA, Tilorone analog R11-877DA, Tilorone analog R10,874DA, and a combination thereof. In certain aspects, Parthenolide and the second agent are in a mixture ore are housed separately. In particular embodiments, the composition comprises Parthenolide and at least one inhibitor of TLR4/MD-2/CD14; comprises Parthenolide and Resatorvid; comprises Parthenolide and Curcumin; comprises Parthenolide and one or more nACHR agonists; comprises Parthenolide and Tilorone or Parthenolide and at least one Tilorone analog; and/or comprises Parthenolide and a neurodegenerative disease treatment, such as an Alzheimer's Disease treatment.

In specific embodiments of the invention, the ratio of Parthenolide to a second agent in the composition is 1:1, 1:2, 1:10, 1:50, 1:100, 1:500, 1:1000, 2:1, 10:1, 50:1, 100:1, 500:1, or 1000:1. In some aspects, the composition has a form that is a tablet, liquid, lozenge, injectable composition, or dissolvable film. In some cases, Parthenolide and the second agent are of the same form or are in different forms.

In some embodiments, there is a method of treating a neurodegenerative disease in an individual, comprising the step of delivering to the individual a therapeutically effective amount of a composition of the invention. In some embodiments, a method further comprises the step of delivering to the individual an additional neurodegenerative disease treatment. In some cases, Parthenolide and the second agent are delivered concomitantly to the individual or are delivered at separate times to the individual. The composition may be delivered orally, subcutaneously, intramuscularly, or intravenously, in specific embodiments. Any two compositions may be delivered to an individual via separate delivery routes or the same delivery route, and the timing may or may not be simultaneous. In specific embodiments, the composition is delivered to the individual more than once. In some cases, the composition is delivered to the individual at least once daily. In specific embodiments, the composition is delivered to the individual more than once a day, more than once a week, once a week, once a month, or once a year. In particular aspects, a method further comprises the step of diagnosing neurodegenerative disease in the individual.

In some embodiments, there is a kit comprising a composition of the invention, said composition housed in a suitable container or in suitable containers.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### BRIEF DESCRIPTION OF THE DRAWINGS

For a more complete understanding of the present invention, reference is now made to the following descriptions taken in conjunction with the accompanying drawing, in which:

FIG. 1 is a graphical representation of an exemplary methodology used to identify a drug for the treatment of AD.

FIG. 2 shows a scheme illustrating the potential pathway of inflammatory response in microglia (from Burguillos et al. Nature, 472, 319-324, 2011).

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

### I. General Embodiments of the Invention

In general embodiments of the invention, there are combinatorial compositions that comprise Parthenolide and at least one other compound for the treatment of a NDD, such as AD. The compositions may be provided to an individual for prevention or delay of AD, including in some embodiments delaying the onset of AD until a later age or preventing the onset of AD. In alternative embodiments, however, the compositions alleviate or eradicate at least one symptom of AD. In some cases, both improvement of at least one symptom and effective delay of onset of AD is achieved with methods and compositions of the invention.

In some embodiments of the invention, an individual is treated that is 65 years of age or older, although in certain embodiments the individual has early onset (also known as younger-onset) AD, such as an individual in their 40's or 50's. In some aspects, the individual has familiar AD.

The present invention employs Parthenolide and another or more agent(s) for a synergistic or, in some cases, additive, effect to improve or decrease the rate of neurodegeneration in an individual in need thereof.

Although Parthenolide is generally recognized as an apoptosis inducer, in some embodiments of the invention Parthenolide inhibits the proapoptotic function of NF-κB and, consequently, apoptosis, rather than inducing apoptosis in microglia. In some embodiments of the invention, Parthenolide acts as a proapoptotic inhibitor and is combined with other agent(s), such as inhibitor(s) of the tri-molecular complex (TLR4/MD-2/CD14) (including Resatorvid and/or Curcumin or a combination thereof) and/or nACHR agonist(s) including Tilorone or Tilorone analog, or a combination thereof.

### II. Treatment of Alzheimer's Disease

Currently, no therapy has been developed that will prevent or delay AD progression (Roberson and Mucke 2006). Present therapies treat one or more symptoms of AD, including memory loss that disrupts daily life; challenges in planning or solving problems, difficulty completing familiar tasks at home, at work or at leisure, confusion with time or place, trouble understanding visual images and spatial relationships, new problems with words in speaking or writing, misplacing things and losing the ability to retrace steps, decreased or poor judgment, withdrawal from work or social activities, changes in mood and personality (Alzheimer's Association).

The currently available symptomatic therapies for AD mildly improve defects in cognitive function, activities of daily living (ADLs) and global functioning (Mangialasche, et al 2011). United States Food and Drug Administration (FDA) approved drugs for the treatment of AD includes memantine, galantamine, rivastigmine and donepezil, albeit they are not curative.

Drugs that treat the symptoms of AD based on the enhancement of neurotransmitter systems include the acetylcholinesterase (AChE) inhibitors (donepezil, galantamine and rivastigmine) that reduce the enzymatic degradation of the neurotransmitter ACh, thus enhancing the cholinergic system in the AD brain. These three AChE inhibitors improve cognition, function in ADL, and behavior in patients with AD (Doody, et al 2001, Roberson and Mucke 2006) and are most effective in treating mild to moderate AD (Geldmacher 2004, Geldmacher 2008). Another drug that treats the symptoms of AD based on the enhancement of neurotransmitter systems is the N-methyl-d-aspartate (NMDA) receptor antagonist, memantine. Memantine is the first FDA approved drug for the treatment of moderate to severe AD (Witt, et al 2004) and has been demonstrated to improve cognitive function (Atri, et al 2008). Additionally, patients with moderate to severe AD treated with memantine in combination with the AChE inhibitors (donepezil, galantamine, or rivastigmine) significantly slowed the deterioration in both cognitive function and ADLs compared to patients treated with the AChE inhibitors alone (Atri, et al 2008).

Thus, in certain aspects, cholinesterase inhibitors (Aricept, Exelon, Razadyne, Cognex) and/or memantine (Namenda) are employed to address the cognitive symptoms (memory loss, confusion, and problems with thinking and reasoning) of Alzheimer's disease.

In some cases, individuals utilize herbal remedies, dietary supplements or medical foods, such as one or more of the following: caprylic acid; coconut oil; coenzyme Q10; coral calcium; ginkgo biloba; Huperzine A; Omega-3 fatty acids; phosphatidylserine; and/or tramiprosate.

Any of the aforementioned compounds may be employed in embodiments of the present invention.

In some embodiments of the invention, an individual suspected of having or known to have AD or an individual that is 65 years of age or older is subjected to methods and compositions of the invention. In particular embodiments, an individual is also subjected to diagnosis of AD, which may include a thorough medical history; mental status testing; a physical and neurological exam; and/or tests (such as blood tests and brain imaging) to rule out other causes of dementia-like symptoms.

### III. Pharmaceutical Preparations

Pharmaceutical compositions of the present invention comprise an effective amount of one or more Parthenolide/second agent compositions dissolved or dispersed in a pharmaceutically acceptable carrier. When Parthenolide and the second agent are housed and/or delivered separately, they each are dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one Parthenolide combinatorial composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The Parthenolide combinatorial composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990 incorporated herein by reference).

The Parthenolide combinatorial composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the Parthenolide combinatorial composition, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the Parthenolide combinatorial composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the Parthenolide combinatorial composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the Parthenolide combinatorial composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable fix intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound Parthenolide combinatorial composition may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and Parthenolide combinatorial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Kits of the Invention

Any of the compositions described herein may be comprised in a kit. The kits will thus comprise, in suitable container means, a Parthenolide combinatorial composition of the present invention. In some embodiments, the kit further comprises an additional agent for treating a microbial infection, and the additional agent may be combined with the composition of the invention or may be provided separately in the kit. In some embodiments, means of taking a sample from an individual and/or of assaying the sample may be provided in the kit. In certain embodiments there may be means to identify AD in an individual and/or an additional neurodegenerative disease treatment.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (for example, when Parthenolide and the second agent are housed separately), the kit also will generally contain a second, third or other additional container into which the additional components may be separately placed. However, various combinations of components may be comprised in a vial. The kits of the present invention also will typically include a means for containing the Parthenolide combinatorial composition and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are offered by way of example and are not intended to limit the scope of the invention in any manner.

### Example 1

### Identification of Parthenolide and a Second Agent for AD Treatment

Due to the high morbidity and sequelae of AD, a Literature Based Discovery (LBD) approach was designed to pinpoint a candidate drugs for the treatment of AD that may be more suitable treatment than the currently administered regimen.

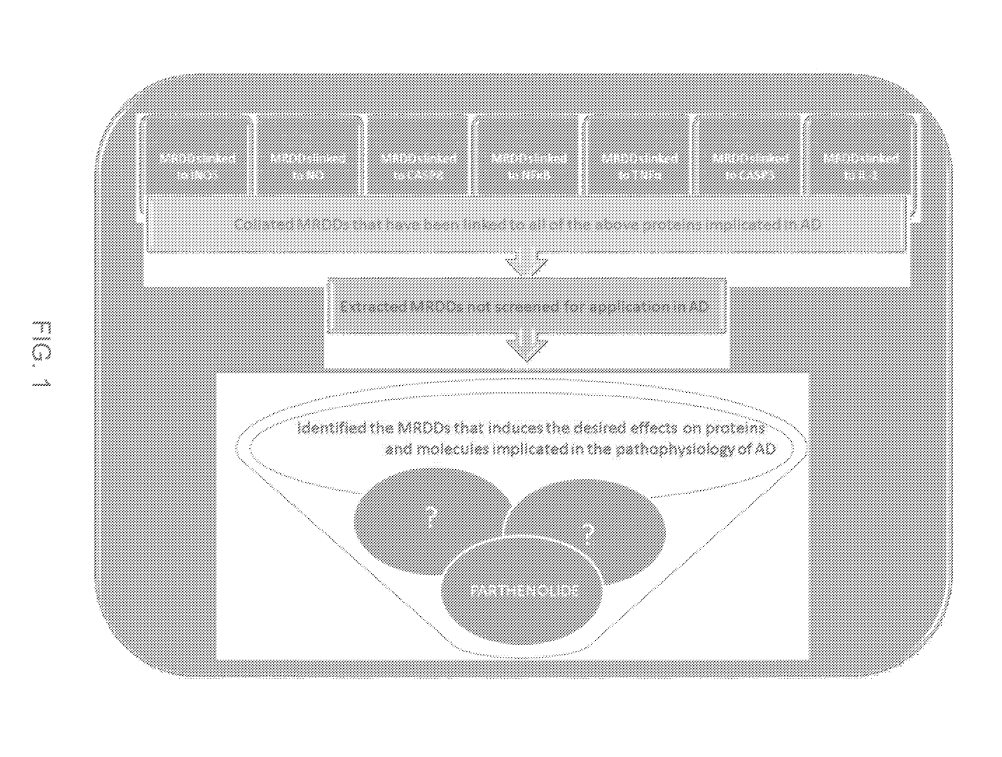

Exemplary Methodology (FIG. 1):

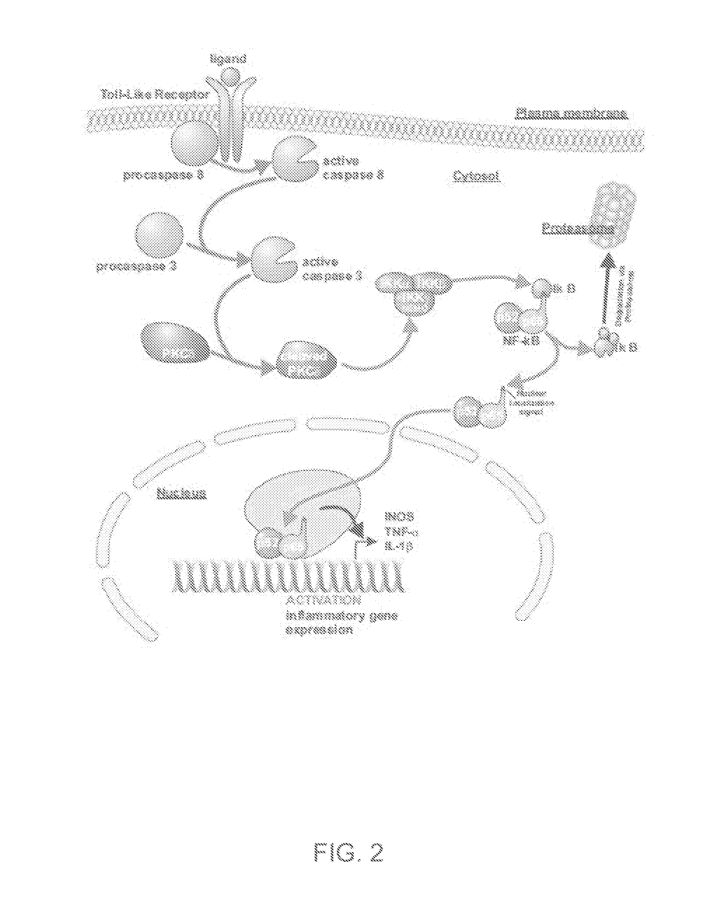

1. Dragon Exploratory System an in house fact-finding system for biomedical and biology domain that operates mainly based on LBD, and was used to generate a knowledgebase on neurodegenerative diseases. This knowledge base is focused on neurodegenerative disease-related literature that allowed extraction of microglia-related disease drugs (MRDDs) linked to specific genes and proteins implicated in the pathophysiology of AD (FIG. 2).

2. The MRDDs that were linked to all of the selected AD-related proteins were compiled.

3. The MRDDs that were not screened for application as an AD drug were extracted.

4. The MRDDs from step 3 that induce the desired effect on the molecules and proteins implicated in the pathophysiology of AD were selected by hand curation.

The study allowed for the identification of Parthenolide as a critical component in the newly proposed treatment of AD. Parthenolide, a known NF-κB inhibitor, is a naturally occurring sesquiterpene lactone derived from feverfew (Tanacetum parthenium) (Mathema, et al 2011).

In 2002, Fiebich et al. demonstrated that parthenolide inhibits INOS/NO synthesis in primary rat microglia (Fiebich, et al 2002) (Table 1). This is the only experimental data published demonstrating the effect of parthenolide in microglia. However, Parthenolide has additionally been shown to inhibit IkappaBalpha degradation, NF-κB activation and inflammatory response in IL-1β and TNFα-stimulated cystic fibrosis cells (Saadane, et al 2007). Thus, Parthenolide also inhibits the reactivation of NF-κB by inflammatory proteins implicated in the pathophysiology of AD. Mangolini et al. further demonstrated that Parthenolide reduces, rather than increase apoptosis and p53 levels in fibrocystin/polyductin-depleted kidney epithelial cells (Mangolini, et al 2010). This result highlights the proapoptotic function of NF-κB in particular cell types and Parthenolide's ability to reduce apoptosis via NF-κB inhibition. Consequently, in embodiments of the invention Parthenolide also inhibits NF-κB and the associated pro-inflammatory signals in microglia, thereby reducing neurodegeneration in AD.

TABLE 1
Beneficial effects demonstrated by Parthenolide on the
pathophysiology of AD

| | Expression | | Expression altered by |
|---|---|---|---|
| Molecules | status in AD | References | Parthenolide |
| CASP8 | up | | undetermined |
| CASP3 | up | | undetermined |
| IkappaBα | up | (Saadane, et al 2007) | Decreases IkappaBα |
| | | | levels |
| NF-κB | up | (Saadane, et al 2007) | Decreases NF-κB |
| | | | levels |
| NO | up | (Fiebich, et al 2002) | Decreases NO levels |
| INOS | up | (Fiebich, et al 2002) | Decreases INOS |
| | | | levels |
| TNFα | up | (Saadane, et al 2007) | Decreases TNFα |
| | | | levels |
| IL-1β | up | (Saadane, et al 2007) | Decreases IL-1β |
| | | | levels |
| TLR4 | up | | undetermined |
| MD-2 | up | | undetermined |
| CD14 | up | | undetermined |

However, this neurodegeneration can be further limited by introducing inhibitors of the tri-molecular receptor complex (TLR4/MD-2/CD14) necessary for full cellular activation by aggregated amyloid peptide. As an example, Curcumin has been demonstrated to bind MD-2 thereby inhibiting MyD88-dependent and -independent signaling pathways of LPS signaling through TLR4 (Gradisar, et al 2007).

On the other hand, Resatorvid (TAK-242), a novel synthetic small-molecule was shown to suppress TLR4 signaling by binding directly to a specific amino acid Cys747 in the intracellular domain of TLR4 (Takashima, et al 2009). Resatorvid was further shown to inhibit TIRAP-mediated activation of NF-κB and the TRAM-mediated activation of NF-κB and interferon-sensitive response element in HEK293 cells stably expressing TLR4/MD-2/CD14 (Matsunaga, et al 2011), Caspase-8 and -3 was shown to be activated in microglia in the brain of individuals with PD and AD, whilst the knockdown of TLR4 was shown to reduce processing/activation of caspase-8 and -3 (Burguillos, et al 2011). Since caspase-8 and -3 processing/activation is reduce and not eliminated, it is possible that other undefined pathways may likely be induced by the Aβ peptides as well. Thus, combining Curcumin that binds MD-2 and Resatorvid that binds TLR4 to inhibit the known pathways induced by Aβ peptides with parthenolide inhibiting all inflammatory pathways likely merging at NF-κB should provide a stronger defense against the effects of the Aβ peptides. Additionally, nAChR agonist/s such as Tilorone (or Tilorone analog's) (Briggs, et al 2008) can also be used to help restore normal cellular processes, as it has been demonstrated that nAChR agonists increase sAPPα secretion whilst decreasing levels of Aβ peptides (Mousavi and Hellstrom-Lindahl 2009).

Considering that no therapy has been developed that will prevent or delay AD progression and that the currently available symptomatic therapies for AD are only mildly improving defects in cognitive function, HDLs and global functioning, it would be beneficial to include Parthenolide and the inhibitor/s of the trimolecular receptor complex (TLR4/MD-2/CD14) such as Curcumin and/or Resatorvid, and the nACHR agonist/s such as Tilorone to the regimen as this drug synergy approach is efficient in changing the progression of AD, in certain aspects of the invention.

### Example 2

### Combinatorial Drugs for Neurodegenerative Disease Treatment

In at least certain embodiments of the invention that employ parthenolide in combination with other compositions for neurodegenerative disease, such combinations are beneficial compared to the current treatment of AD. In particular embodiments, the methods of the invention are directly or indirectly associated with advantages such as decreasing levels of Aβ peptides, for example through inhibiting the action of Aβ peptides via toll-like receptors, nAChR and other inflammatory pathways related to NF-κB activities. In some embodiments of the invention, there are methods and combination that reduce neuronal loss.

In combinatorial treatment aspects of the invention, neurodegeneration can be further limited by introducing inhibitors of the tri-molecular receptor complex (TLR4/MD-2/CD14) necessary for full cellular activation by aggregated amyloid peptide. As an example, Curcumin has been demonstrated to bind MD-2 thereby inhibiting MyD88-dependent and -independent signaling pathways of LPS signaling through TLR4 (Gradisar, et al 2007). On the other hand, Resatorvid (TAK-242), a novel synthetic small-molecule was shown to suppress TLR4 signaling by binding directly to a specific amino acid Cys747 in the intracellular domain of TLR4 (Takashima, et al 2009).

Resatorvid was further shown to inhibit TIRAP-mediated activation of NF-κB and the TRAMmediated activation of NF-κB and interferon-sensitive response element in HEK293 cells stably expressing TLR4/MD-2/CD14 (Matsunaga, et al 2011). Caspase-8 and -3 was shown to be activated in microglia in the brain of individuals with PD and AD, whilst the knockdown of TLR4 was shown to reduce processing/activation of caspase-8 and -3 (Burguillos, et al 2011). Since caspase-8 and -3 processing/activation is reduce and not eliminated, it is possible that other undefined pathways may likely be induced by the Aβ peptides as well.

Thus, combining Curcumin that binds MD-2 and Resatorvid that binds TLR4 to inhibit the known pathways induced by Aβ peptides with Parthenolide inhibiting all inflammatory pathways likely involving NF-κB should provide a stronger defense against the effects of the Aβ peptides. Additionally, nAChR agonists such as Tilorone (or Tilorone analog/s) (Briggs, et al 2008) can also be used to help restore normal cellular processes, as it has been demonstrated that nAChR agonists increase sAPPα secretion whilst decreasing levels of Aβ peptides (Mousavi and Hellstrom-Lindahl 2009).

Considering that no therapy has been developed that will prevent or delay AD progression, and the currently available symptomatic therapies for AD are only mildly improving defects in cognitive function, ADLs and global functioning, it is beneficial to include Parthenolide and at least one other compound, such as the inhibitor(s) of the tri-molecular receptor complex (TLR4/MD-2/CD14), such as Resatorvid, or both Resatorvid and Curcumin, and the nACHR agonist/s such as Tilorone to the regimen. In particular embodiments, this drug synergy approach is efficient to improve at least one symptom of at least one neurodegenerative disease and, in at least some aspects, is efficient to change the progression of AD.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

Acker, B. A., Jacobsen, E. J., Rogers, B. N., Wishka, D. G., Reitz, S. C., Piotrowski, D. W., Myers, J. K., Wolfe, M. L., Groppi, V. E., Thornburgh, B. A., Tinholt, P. M., Walters, R. R., Olson, B. A., Fitzgerald, L., Staton, B. A., Raub, T. J., Krause, M., Li, K. S., Hoffmann, W. E., Hajos, M., Hurst, R. S. & Walker, D. P. (2008) Discovery of N-[(3R,5R)-1-azabicyclo[3,2.1]oct-3-yl]furo[2,3-c]pyridine-5-carboxamide as an agonist of the alpha7 nicotinic acetylcholine receptor: in vitro and in vivo activity. Bioorg Med Chem Lett, 18, 3611-3615.

Ahn, S. I., Lee, J. K. & Youn, H. S. (2009) Inhibition of homodimerization of toll-like receptor 4 by 6-shogaol, Mol Cells, 27, 211-215.

Ajamieh, H., Farrell, G., Wong, H. J., Yu, J., Chu, E., Chen, J. & Teoh, N. (2012) Atorvastatin protects obese mice against hepatic ischemia-reperfusion injury by TLR4 suppression and eNOS activation. J. Gastroenterol Hepatol.

Aliprantis, A. O., Yang, R. B., Weiss, D. S., Godowski, P. & Zychlinsky, A. (2000) The apoptotic signaling pathway activated by Toll-like receptor-2. Embo J, 19, 3325-3336.

Atri, A., Shaughnessy, L. W., Locascio, J. J. & Growdon, J. H. (2008) Long-term course and effectiveness of combination therapy in Alzheimer disease. Alzheimer Dis Assoc Disord, 22, 209-221.

Babic, T. (1999) The cholinergic hypothesis of Alzheimer's disease: a review of progress. J Neurol Neurosurg Psychiatry, 67, 558.

Boess, F. G., De Vry, J., Erb, C., Flessner, T., Hendrix, M., Luithle, J., Methfessel, C., Riedl, B., Schnizler, K., van der Staay, F. J., van Kampen, M., Wiese, W. B. & Koenig, G. (2007) The novel alpha7 nicotinic acetylcholine receptor agonist N-[(3R)-1-azabicyclo[2.2.2]oct-3-yl]-7-[2-(methoxy)phenyl]-1-benzofuran-2-carboxamide improves working and recognition memory in rodents. J Pharmacol Exp Ther, 321, 716-725.

Bredesen, D. E., Rao, R. V. & Mehlen, P. (2006) Cell death in the nervous system. Nature, 443, 796-802.

Briggs, C. A., Schrimpf, M. R., Anderson, D. J., Gubbins, E. J., Gronlien, J. H., Hakerud, M., Ween, H., Thorin-Hagene, K., Malysz, J., Li, J., Bunnelle, W. H., Gopalakrishnan, M. & Meyer, M. D. (2008) alpha7 nicotinic acetylcholine receptor agonist properties of tilorone and related tricyclic analogues. Br J Pharmacol, 153, 1054-1061.

Brown, G. C. & Bal-Price, A. (2003) Inflammatory neurodegeneration mediated by nitric oxide, glutamate, and mitochondria. Mol Neurobiol, 27, 325-355.

Burguillos, M. A., Deierborg, T., Kavanagh, E., Persson, A., Hajji, N., Garcia-Quintanilla, A., Cano, J., Brandin, P., Englund, E., Venero, J. L. & Joseph, B. (2011) Caspase signaling controls microglia activation and neurotoxicity. Nature, 472, 319-324.

Colton, C. A., Snell, J., Chernyshev, O. & Gilbert, D. L. (1994) Induction of superoxide anion and nitric oxide production in cultured microglia. Ann N Y Acad Sci, 738, 54-63.

Cummings, J. L., Vinters, H. V., Cole, G. M. & Khachaturian, Z. S. (1998) Alzheimer's disease: etiologies, pathophysiology, cognitive reserve, and treatment opportunities. Neurology, 51, S2-17; discussion S65-17.

Datla, P., Kalluri, M. D., Basha, K., Bellary, A., Kshirsagar, R., Kanekar, Y., Upadhyay, S., Singh, S. & Rajagopal, V. (2010) 9,10-dihydro-2,5-dimethoxyphenanthrene-1,7-diol, from Eulophia ochreata, inhibits inflammatory signaling mediated by Toll-like receptors. Br J Pharmacol 160, 1158-1170.

Doody, R. S., Stevens, J. C., Beck, C., Dubinsky, R. M., Kaye, J. A., Gwyther, L., Mohs, R. C., Thal, L. J., Whitehouse, P. J., DeKosky, S. T. & Cummings, J. L. (2001) Practice parameter: management of dementia (an evidence-based review). Report of the Quality Standards Subcommittee of the American Academy of Neurology. Neurology, 56, 1154-4166.

Du, R. H., Li, E. G., Cao, Y., Song, Y. C. & Tan, R. X. (2011) Fumigaclavine C inhibits tumor necrosis factor alpha production via suppression of toll-like receptor 4 and nuclear factor kappaB activation in macrophages. Life Sci, 89, 235-240.

Duan, G. J., Zhu, J., Wan, J. Y., Li, X., Ge, X. D., Liu, L. M. & Liu, Y. S. (2010) A synthetic MD 2 mimetic peptide attenuates lipopolysaccharide-induced inflammatory responses in vivo and in vitro. Int Immunopharmacol, 10, 1091-1100.

Ekshyyan, O. & Aw, T. Y. (2004) Apoptosis: a key in neurodegenerative disorders. Curr Neurovasc Res, 1, 355-371.

Fiebich, B. L., Lieb, K., Engels, S. & Heinrich, M. (2002) Inhibition of LPS-induced p42/44 MAP kinase activation and iNOS/NO synthesis by parthenolide in rat primary microglial cells. J Neuroimmunol, 132, 18-24.

Geldmacher, D. S. (2004) Donepezil (Aricept) for treatment of Alzheimer's disease and other dementing conditions. Expert Rev Neurother, 4, 5-16.

Geldmacher, D. S. (2008) Cost-effectiveness of drug therapies for Alzheimer's disease: A brief review. Neuropsychiatr Dis Treat, 4, 549-555.

Ghiron, C., Haydar, S. N., Aschmies, S., Bothmann, H., Castaldo, C., Cocconcelli, G., Comery, T. A., Di, L., Dunlop, J., Lock, T., Kramer, A., Kowal, D., Jow, F., Grauer, S., Harrison, B., La Rosa, S., Maccari, L., Marquis, K. L., Micco, I., Nencini, A., Quinn, J., Robichaud, A. J., Roncarati, R., Scali, C., Terstappen, G. C., Turlizzi, E., Valacchi, M., Varrone, M., Zanaletti, R. & Zanelli, U. (2010) Novel alpha-7 nicotinic acetylcholine receptor agonists containing a urea moiety: identification and characterization of the potent, selective, and orally efficacious agonist 1-[6-(4-fluorophenyl)pyridin-3-yl]-3-(4-piperidin-1-ylbutyl)urea (SEN34625/WYE-103914). J Med Chem, 53, 4379-4389.

Giulian, D. (1987) Ameboid microglia as effectors of inflammation in the central nervous system. J Neurosci Res, 18, 155-171, 132-153.

Goedert, M. & Spillantini, M. G. (2006) A century of Alzheimer's disease. Science, 314, 777-781.

Gradisar, H., Keber, M. M., Pristovsek, P. & Jerala, R. (2007) MD-2 as the target of curcumin in the inhibition of response to LPS. J Leukoc Biol, 82, 968-974.

Griffin, W. S., Stanley, L. C., Ling, C., White, L., MacLeod, V., Perrot, L. J., White, C. L., 3rd & Araoz, C. (1989) Brain interleukin 1 and S-100 immunoreactivity are elevated in Down syndrome and Alzheimer disease. Proc Natl Acad Sci USA, 86, 7611-7615.

Hajos, M., Hurst, R. S., Hoffmann, W. E., Krause, M., Wall, T. M., Higdon, N. R. & Groppi, V. E. (2005) The selective alpha7 nicotinic acetylcholine receptor agonist PNU-282987 [N-[(3R)-1-Azabicyclo[2.2.2]oct-3-yl]-4-chlorobenzamide hydrochloride] enhances GABAergic synaptic activity in brain slices and restores auditory gating deficits in anesthetized rats. J Pharmacol Exp Ther, 312, 1211-4222.

Hardy, J. & Selkoe, D. J. (2002) The amyloid hypothesis of Alzheimer's disease: progress and problems on the road to therapeutics. Science, 297, 353-356.

Haydar, S. N., Ghiron, C., Bettinetti, L., Bothmann, H., Comery, T. A., Dunlop, J., La Rosa, S., Micco, I., Pollastrini, M., Quinn, J., Roncarati, R., Scali, C., Valacchi, M., Varrone, M. & Zanaletti, R. (2009) SAR and biological evaluation of SEN12333/WAY-317538: Novel alpha 7 nicotinic acetylcholine receptor agonist. Bioorg Med Chem, 17, 5247-5258.

Ibrahim, A., Mbodji, K., Hassan, A., Aziz, M., Boukhettala, N., Coeffier, M., Savoye, G., Dechelotte, P. & Marion-Letellier, R. (2011) Anti-inflammatory and anti-angiogenic effect of long chain n-3 polyunsaturated fatty acids in intestinal microvascular endothelium. Clin Nutr, 30, 678-687.

Isacson, O., Seo, H., Lin, L., Albeck, D. & Granholm, A. C. (2002) Alzheimer's disease and Down's syndrome: roles of APP, trophic factors and ACh. Trends Neurosci, 25, 79-84.

Itagaki, S., McGeer, P. L., Akiyama, H., Zhu, S. & Selkoe, D. (1989) Relationship of microglia and astrocytes to amyloid deposits of Alzheimer disease. J Neuroimmunol, 24, 173-182.

Kim, H. Y., Park, J., Lee, K. H., Lee, D. U., Kwak, J. H., Kim, Y. S. & Lee, S. M. (2011) Ferulic acid protects against carbon tetrachloride-induced liver injury in mice. Toxicology, 282, 104-111.

Kitazawa, T., Tsujimoto, T., Kawaratani, H. & Fukui, H. (2010) Salvage effect of E5564, Toll-like receptor 4 antagonist on d-galactosamine and lipopolysaccharide-induced acute liver failure in rats. J Gastroenterol Hepatol, 25, 1009-1012.

Lee, J. S., Lee, J. Y., Lee, M. Y., Hwang, D. H. & Youn, H. S. (2008) Acrolein with an alpha, beta-unsaturated carbonyl group inhibits LPS-induced homodimerization of toll-like receptor 4. Mol Cells, 25, 253-257.

Liu, L., Ghosh, N., Slivka, P. F., Fiorini, Z., Hutchinson, M. R., Watkins, L. R. & Yin, H. (2011) An MD2 hot-spot-mimicking peptide that suppresses TLR4-mediated inflammatory response in vitro and in vivo. Chembiochem, 12, 1827-1831.

Liu, Q., Huang, Y., Xue, F., Simard, A., DeChon, J., Li, G., Zhang, J., Lucero, L., Wang, M., Sierks, M., Hu, G., Chang, Y., Lukas, R. J. & Wu, J. (2009) A novel nicotinic acetylcholine receptor subtype in basal forebrain cholinergic neurons with high sensitivity to amyloid peptides, J Neurosci, 29, 918-929.

Liu, Q., Zerbinatti, C. V., Zhang, J., Hoe, H. S., Wang, B., Cole, S. L., Herz, J., Muglia, L. & Bu, G. (2007) Amyloid precursor protein regulates brain apolipoprotein E and cholesterol metabolism through lipoprotein receptor LRP1. Neuron, 56, 66-78.

Malysz, J., Anderson, D. J., Gronlien, J. H., Ji, J., Bunnelle, W. H., Hakerud, M., Thorin-Hagene, K., Ween, H., Helfrich, R., Hu, M., Gubbins, E., Gopalakrishnan, S., Puttfarcken, P. S., Briggs, C. A., Li, J., Meyer, M. D., Dyhring, T., Ahring, P. K., Nielsen, E. O., Peters, D., Timmermann, D. B. & Gopalakrishnan, M. (2010) In vitro pharmacological characterization of a novel selective alpha7 neuronal nicotinic acetylcholine receptor agonist ABT-107. J Pharmacol Exp Ther, 334, 863-874.

Malysz, J., Gronlien, J. H., Anderson, D. J., Hakerud, M., Thorin-Hagene, K., Ween, H., Wetterstrand, C., Briggs, C. A., Faghih, R., Bunnelle, W. H. & Gopalakrishnan, M. (2009) In vitro pharmacological characterization of a novel allosteric modulator of alpha 7 neuronal acetylcholine receptor, 4-(5-(4-chlorophenyl)-2-methyl-3-propionyl-1H-pyrrol-1-yl)benzenesulfonamide (A-867744), exhibiting unique pharmacological profile. J Pharmacol Exp Ther, 330, 257-767.

Mangialasche, F., Solomon, A., Winblad, B., Mecocci, P. & Kivipelto, M. (2011) Alzheimer's disease: clinical trials and drug development. Lancet Neurol, 9, 702-716.

Mangolini, A., Bogo, M., Durante, C., Borgatti, M., Gambari, R., Harris, P. C., Rizzuto, R., Pinton, P., Aguiari, G. & del Senno, L. (2010) NF-kappaB activation is required for apoptosis in fibrocystin/polyductin-depleted kidney epithelial cells. Apoptosis, 15, 94-104.

Marquis, K. L., Comery, T. A., Jow, F., Navarra, R. L., Grauer, S. M., Pulicicchio, C., Kelley, C., Brennan, J. A., Roncarati, R., Scali, C., Haydar, S., Ghiron, C., Terstappen, G. C. & Dunlop, J. (2011) Preclinical assessment of an adjunctive treatment approach for cognitive impairment associated with schizophrenia using the alpha7 nicotinic acetylcholine receptor agonist WYE-103914/SEN34625. Psychopharmacology (Berl), 218, 635-647.

Marzolo, M. P., von Bernhardi, R. & Inestrosa, N. C. (1999) Mannose receptor is present in a functional state in rat microglia cells. J Neurosci Res, 58, 387-395.

Mathema, V. B., Koh, Y. S., Thakuri, B. C. & Sillanpaa, M. (2011) Parthenolide, a Sesquiterpene Lactone, Expresses Multiple Anti-cancer and Anti-inflammatory Activities. Inflammation, [Epub ahead of print].

Matsunaga, N., Tsuchimori, N., Matsumoto, T. & Ii, M. (2011) TAK-242 (resatorvid), a small-molecule inhibitor of Toll-like receptor (TLR) 4 signaling, binds selectively to TLR4 and interferes with interactions between TLR4 and its adaptor molecules. Mol Pharmacol, 79, 34-41.

Mita, S., Shimizu, Y., Notsu, T., Imada, K. & Kyo, S. (2011) Dienogest inhibits Toll-like receptor 4 expression induced by costimulation of lipopolysaccharide and high-mobility group box 1 in endometrial epithelial cells. Fertil Steril, 96, 1485-1489 e1484,

Moses, G. S., Jensen, M. D., Lue, L. F., Walker, D. G., Sun, A. Y., Simonyi, A. & Sun, G. Y. (2006) Secretory PLA2-IIA: a new inflammatory factor for Alzheimer's disease, J Neuroinflammation, 3, 28.

Mousavi, M. & Hellstrom-Lindahl, E. (2009) Nicotinic receptor agonists and antagonists increase sAPPalpha secretion and decrease Abeta levels in vitro. Neurochem Int, 54, 237-244.

Mousavi, M., Hellstrom-Lindahl, E., Guan, Z. Z., Shan, K. R., Ravid, R. & Nordberg, A. (2003) Protein and mRNA levels of nicotinic receptors in brain of tobacco using controls and patients with Alzheimer's disease. Neuroscience, 122, 515-520.

Murakami, K., Tanaka, M., Usui, T., Kawabata, D., Shiomi, A., Iguchi-Hashimoto, M., Shimizu, M., Yukawa, N., Yoshifuji, H., Nojima, T., Ohmura, K., Fujii, T., Umehara, H. & Mimori, T. (2012) Follistatin-related protein/follistatin-like 1 evokes an innate immune response via CD14 and toll-like receptor 4. FEBS Lett, 586, 319-324.

Nagahara, Y., Nagamori, T., Tamegai, H., Hitokuwada, M., Yoshimi, Y., Ikekita, M. & Shinomiya, T. (2011) Inulin stimulates phagocytosis of PMA-treated THP-1 macrophages by involvement of PI3-kinases and MAP kinases. Biofactors, 37, 447-454.

O'Donnell, C. J., Rogers, B. N., Bronk, B. S., Bryce, D. K., Coe, J. W., Cook, K. K., Duplantier, A. J., Evrard, E., Hajos, M., Hoffmann, W. E., Hurst, R. S., Maklad, N., Mather, R. J., McLean, S., Nedza, F. M., O'Neill, B. T., Peng, L., Qian, W., Rottas, M. M., Sands, S. B., Schmidt, A. W., Shrikhande, A. V., Spracklin, D. K., Wong, D. F., Zhang, A. & Zhang, L. (2010) Discovery of 4-(5-methyloxazolo[4,5-b]pyridin-2-yl)-1,4-diazabicyclo[3.2.2]nonane (CP-810,123), a novel alpha 7 nicotinic acetylcholine receptor agonist for the treatment of cognitive disorders in schizophrenia: synthesis, SAR development, and in vivo efficacy in cognition models. J Med Chem, 53, 1222-1237.

Parihar, M. S. & Hemnani, T. (2004) Alzheimer's disease pathogenesis and therapeutic interventions. J Clin Neurosci, 11, 456-467.

Park, S. E., Sapkota, K., Kim, S., Kim, H. & Kim, S. J. (2011) Kaempferol acts through mitogen-activated protein kinases and protein kinase B/AKT to elicit protection in a model of neuroinflammation in BV2 microglial cells. Br J Pharmacol, 164, 1008-1025.

Park, S. J. & Youn, H. S. (2010) Suppression of homodimerization of toll-like receptor 4 by isoliquiritigenin. Phytochemistry, 71, 1736-1740.

Puddu, P., Latorre, D., Carollo, M., Catizone, A., Ricci, G., Valenti, P. & Gessani, S. (2011) Bovine lactoferrin counteracts Toll-like receptor mediated activation signals in antigen presenting cells. PLoS One. 6, e22504.

Riehl, T. E., Foster, L. & Stenson, W. F. (2012) Hyaluronic acid is radioprotective in the intestine through a TLR4 and COX-2-mediated mechanism. Am J Physiol Gastrointest Liver Physiol, 302, G309-316.

Roberson, E. D. & Mucke, L. (2006) 100 years and counting: prospects for defeating Alzheimer's disease. Science, 314, 781-784.

Rogers, J., Cooper, N. R., Webster, S., Schultz, J., McGeer, P. L., Styren, S. D., Civin, W. H., Brachova, L., Bradt, B., Ward, P. & et al. (1992) Complement activation by beta-amyloid in Alzheimer disease. Proc Natl Acad Sci USA, 89, 10016-10020.

Rogers, J. T., Leiter, L. M., McPhee, J., Cahill, C. M., Zhan, S. S., Potter, H. & Nilsson, L. N. (1999) Translation of the alzheimer amyloid precursor protein mRNA is up-regulated by interleukin-1 through 5′-untranslated region sequences. J Biol Chem, 274, 6421-6431.

Roh, E., Lee, H. S., Kwak, J. A., Hong, J. T., Nam, S. Y., Jung, S. H., Lee, J. Y., Kim, N. D., Han, S. B. & Kim, Y. (2010) MD-2 as the target of nordipid chalcone in the inhibition of endotoxin LPS-induced TLR4 activity. J Infect Dis, 203, 1012-1020.

Roncarati, R., Scali, C., Comery, T. A., Grauer, S. M., Aschmi, S., Bothmann, H., Jow, B., Kowal, D., Gianfriddo, M., Kelley, C., Zanelli, U., Ghiron, C., Haydar, S., Dunlop, J. & Terstappen, G. C. (2009) Precognitive and neuroprotective activity of a novel alpha7 nicotinic acetylcholine receptor agonist for treatment of neurodegenerative and cognitive disorders. J Pharmacol Exp Ther, 329, 459-468.

Saadane, A., Masters, S., DiDonato, J., Li, J. & Berger, M. (2007) Parthenolide inhibits IkappaB kinase, NF-kappaB activation and inflammatory response in cystic fibrosis cells and mice. Am J Respir Cell Mol Biol, 36, 728-736.

Salawu, F. K., Umar, J. T. & Olokoba, A. B. (2011) Alzheimer's disease: A review of recent developments. Ann Afr Med, 10, 73-79.

Scheuner, D., Eckman, C., Jensen, M., Song, X., Citron, M., Suzuki, N., Bird, T. D., Hardy, J., Hutton, M., Kukull, W., Larson, E., Levy-Lahad, E., Viitanen, M., Peskind, E., Poorkaj, P., Schellenberg, G., Tanzi, R., Wasco, W., Lannfelt, L., Selkoe, D. & Younkin, S. (1996) Secreted amyloid betaprotein similar to that in the senile plaques of Alzheimer's disease is increased in vivo by the presenilin 1 and 2 and APP mutations linked to familial Alzheimer's disease. Nat Med, 2, 864-870.

Srivareerat, M., Tran, T. T., Salim, S., Aleisa, A. M. & Alkadhi, K. A. (2011) Chronic nicotine restores normal Abeta levels and prevents short-term memory and E-LTP impairment in Abeta rat model of Alzheimer's disease. Neurobiol Aging, 32, 834-844.

Takashima, K., Matsunaga, N., Yoshimatsu, M., Hazeki, K., Kaisho, T., Uekata, M., Hazeki, O., Akira, S., Iizawa, Y. & Ii, M. (2009) Analysis of binding site for the novel small-molecule TLR4 signal transduction inhibitor TAK-242 and its therapeutic effect on mouse sepsis model. Br J Pharmacol, 157, 1250-1262.

Tatsumi, R., Fujio, M., Satoh, H., Katayama, J., Takanashi, S., Hashimoto, K. & Tanaka, H. (2005) Discovery of the alpha7 nicotinic acetylcholine receptor agonists. (R)-3′-5-Chlorothiophen-2-yl)spiro-1-azabicyclo[2.2.2]octane-3,5′-[1′,3′]oxazolidin-2-one as a novel, potent, selective, and orally bioavailable ligand. J Med Chem, 48, 2678-2686.

Tatsumi, R., Fujio, M., Takanashi, S., Numata, A., Katayama, J., Satoh, H., Shiigi, Y., Maeda, J., Kuriyama, M., Horikawa, T., Murozono, T., Hashimoto, K. & Tanaka, H. (2006) (R)-3′-(3-methylbenzo[b]thiophen-5-yl)spiro[1-azabicyclo[2,2,2]octane-3,5′-oxazolidin]-2′-one, a novel and potent alpha7 nicotinic acetylcholine receptor partial agonist displays cognitive enhancing properties. J Med Chem, 49, 4374-4383.

Tian, J., Wang, Y., He, Z., Gao, Y., Rundhaug, J. E. & Wang, X. (2011) Hydroxyethyl starch (130 kD) inhibits Toll-like receptor 4 signaling pathways in rat lungs challenged with lipopolysaccharide. Anesth Analg, 113, 112-119.

Tidswell, M. & LaRosa, S. P. (2011) Toll-like receptor-4 antagonist eritoran tetrasodium for severe sepsis. Expert Rev Anti Infect Ther, 9, 507-520.

Tu, X. K., Yang, W. Z., Shi, S. S., Chen, Y., Wang, C. H., Chen, C. M. & Chen, Z. (2011) Baicalin inhibits TLR2/4 signaling pathway in rat brain following permanent cerebral ischemia. Inflammation, 34, 463-470.

Tu, X. K., Yang, W. Z., Wang, C. H., Shi, S. S., Zhang, Y. L., Chen, C. M., Yang, Y. K., Jin, C. D. & Wen, S. (2010) Zileuton reduces inflammatory reaction and brain damage following permanent cerebral ischemia in rats. Inflammation, 33, 344-352.

Walter, S., Letiembre, M., Liu, Y., Heine, H., Penke, B., Hao, W., Bode, B., Manietta, N., Walter, J., Schulz-Schafer, W. & Fassbender, K. (2007) Role of the toll-like receptor 4 in neuroinflammation in Alzheimer's disease. Cell Physiol Biochem, 20, 947-956.

Wan, Y., Wu, Y. L., Lian, L. H., Xie, W. X., Li, X., Ouyang, B. Q., Bai, T., Li, Q., Yang, N. & Nan, J. X. (2012) The anti-fibrotic effect of betulinic acid is mediated through the inhibition of NF-kappaB nuclear protein translocation. Chem Biol Interact, 195, 215-223.

Wishka, D. G., Walker, D. P., Yates, K. M., Reitz, S. C., Jia, S., Myers, J. K., Olson, K. L., Jacobsen, E. J., Wolfe, M. L., Groppi, V. E., Hanchar, A. J., Thornburgh, B. A., Cortes-Burgos, L. A., Wong, E. H., Staton, B. A., Raub, T. J., Higdon, N. R., Wall, T. M., Hurst, R. S., Walters, R. R., Hoffmann, W. E., Hajos, M., Franklin, S., Carey, G., Gold, L. H., Cook, K. K., Sands, S. B., Zhao, S. X., Soglia, J. R., Kalgutkar, A. S., Arneric, S. P. & Rogers, B. N. (2006) Discovery of N-[(3R)-1-azabicyclo[2.2.2]oct-3-yl]furo[2,3-c]pyridine-5-carboxamide, an agonist of the alpha7 nicotinic acetylcholine receptor, for the potential treatment of cognitive deficits in schizophrenia: synthesis and structure—activity relationship. J Med Chem, 49, 4425-4436.

Witt, A., Macdonald, N. & Kirkpatrick, P. (2004) Memantine hydrochloride. Nat Rev Drug Discov, 3, 109-110.

Wu, Y., Tang, K., Huang, R. Q., Zhuang, Z., Cheng, H. L., Yin, H. X. & Shi, J. X. (2011) Therapeutic potential of peroxisome proliferator-activated receptor gamma agonist rosiglitazone in cerebral vasospasm after a rat experimental subarachnoid hemorrhage model. J Neurol Sci, 305, 85-91.

Yu, C., Shan, T., Feng, A., Li, Y., Zhu, W., Xie, Y., Li, N. & Li, J. (2011) Triptolide ameliorates Crohn's colitis is associated with inhibition of TLRs/NF-kappaB signaling pathway. Fitoterapia, 82, 709-715.

Zanaletti, R., Bettinetti, L., Castaldo, C., Cocconcelli, G., Comery, T., Dunlop, J., Gaviraghi, G., Ghiron, C., Haydar, S. N., Jow, F., Maccari, L., Micco, I., Nencini, A., Scali, C., Turlizzi, E. & Valacchi, M. (2012) Discovery of a Novel Alpha-7 Nicotinic Acetylcholine Receptor Agonist Series and Characterization of the Potent, Selective, and Orally Efficacious Agonist 5-(4-Acetyl[1,4]diazepan-1-yl)pentanoic Acid [5-(4-Methoxyphenyl)-1H-pyrazol-3-yl]Amide (SEN15924, WAY-361789). J Med Chem.

Zhao, L., Lee, J. Y. & Hwang, D. H. (2011) Inhibition of pattern recognition receptor-mediated inflammation by bioactive phytochemicals. Nutr Rev, 69, 310-320.

One skilled in the art readily appreciates that the present invention is well adapted to carry out the objectives and obtain the ends and advantages mentioned as well as those inherent therein. Methods, procedures, techniques and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

1. A composition comprising Parthenolide and at least a second agent, wherein said second agent is selected from the group consisting of:
a) one or more inhibitors of the tri-molecular receptor complex (TLR4/MD-2/CD14);
b) one or more nicotinic acetylcholine receptors (nACHR) agonists;
c) a neurodegenerative disease treatment; and
d) a combination thereof.

2. The composition of claim 1, wherein an inhibitor of TLR4/MD-2/CD14 is selected from the group consisting of Curcumin, Resatorvid, and a combination thereof.

3. The composition of claim 1, wherein a nACHR agonist is selected from the group consisting of Tilorone, Tilorone analog R11-567DA, Tilorone analog R11-877DA, Tilorone analog R10,874DA, and a combination thereof.

4. The composition of claim 1, wherein Parthenolide and the second agent are in a mixture.

5. The composition of claim 1, wherein Parthenolide and the second agent are housed separately.

6. The composition of claim 1, wherein the composition comprises Parthenolide and at least one inhibitor of TLR4/MD-2/CD14.

7. The composition of claim 1, wherein the composition comprises Parthenolide and Zesatorvid.

8. The composition of claim 1, wherein the composition comprises Parthenolide and Curcumin.

9. The composition of claim 1, wherein the composition comprises Parthenolide and one or more nACHR agonists.

10. The composition of claim 1, wherein the composition comprises Parthenolide and Tilorone or Parthenolide and at least one Tilorone analog.

11. The composition of claim 1, wherein the composition comprises Parthenolide and a neurodegenerative disease treatment.

12. The composition of claim 1, wherein the neurodegenerative disease treatment is an Alzheimer's Disease treatment.

13. The composition of claim 1, wherein the ratio of Parthenolide to the second agent in the composition is 1:1, 1:2, 1:10, 1:50, 1:100, 1:500, 1:1000, 2:1, 10:1, 50:1, 100:1, 500:1, or 1000:1.

14. The composition of claim 1, wherein the composition has a form that is a tablet, liquid, lozenge, injectable composition, or dissolvable film/

15. The composition of claim 1, wherein Parthenolide and the second agent are of the same form.

16. The composition of claim 1, wherein Parthenolide and the second agent are in different forms.

17. A method of treating a neurodegenerative disease in an individual, comprising the step of delivering to the individual a therapeutically effective amount of a composition of claim 1.

18. The method of claim 17, further comprising the step of delivering to the individual an additional neurodegenerative disease treatment.

19. The method of claim 17, wherein Parthenolide and the second agent are delivered concomitantly to the individual.

20. The method of claim 17, wherein Parthenolide and the second agent are delivered at separate times to the individual.

21. The method of claim 17, wherein the composition is delivered orally, subcutaneously, intramuscularly, or intravenously.

22. The method of claim 17, wherein the composition is delivered to the individual more than once.

23. The method of claim 22, wherein the composition is delivered to the individual at least once daily.

24. The method of claim 22, wherein the composition is delivered to the individual more than once a day, more than once a week, once a week, once a month, or once a year.

25. The method of claim 17, further comprising the step of diagnosing neurodegenerative disease in the individual.

26. A kit comprising the composition of claim 1, said composition housed in a suitable container or in suitable containers.
